(19) United States
(12) Patent Application Publication
(10) Pub. No.: US 2014/0171465 A1
(43) Pub. Date: Jun. 19, 2014

(54) Methods For Increasing Reticulocyte Hemoglobin Content

(71) Applicant: Kin-Hung (Peony) Yu, Hillsborough, CA (US)

(72) Inventor: Kin-Hung (Peony) Yu, Hillsborough, CA (US)

(73) Assignee: FibroGen, Inc., San Francisco, CA (US)

(21) Appl. No.: 13/978,163

(22) PCT Filed: Jan. 13, 2012
(86) PCT No.: PCT/US12/21363

§ 371 Date: Nov. 15, 2013

Related U.S. Application Data
(60) Provisional application No. 61/461,094, filed on Jan. 13, 2011.

(51) Int. Cl. A61K 31/472

(52) U.S. Cl. CPC A61K 31/472
USPC 514/309

(57) ABSTRACT

The invention relates to methods and compounds useful for treating deficiencies in hemoglobin production. Methods and compounds useful for increasing reticulocyte hemoglobin content are provided.

### FIELD OF THE INVENTION

The invention relates to methods and compounds useful for treating deficiencies in hemoglobin production. Methods and compounds useful for increasing reticulocyte hemoglobin content are provided.

### BACKGROUND OF THE INVENTION

Effective treatment of anemia in human subjects requires a coordinated response that overcomes pathophysiological stresses antagonizing red blood cell (RBC) production to yield a sufficient population of normal RBCs that can be maintained over time. Anemia typically results from any assault on the function of the kidney or bone marrow as these organs are the primary signal for and production site of, respectively, new RBCs. Thus, patients having kidney dysfunction, such as chronic kidney disease, often have anemia increasing in severity with the degree of dysfunction. Anemia can also result from or be further antagonized by an inflammatory response or infection due to the body's sequestration of iron into storage sites, making it unavailable for hemoglobin production.

Erythropoiesis-stimulating agents (ESAs) are currently used to treat anemia in human patients. The most common ESAs are recombinant human erythropoietins (rhEPO) such as Epogen and Aranesp (Amgen; Thousand Oaks, Calif.). Although ESAs have been used for treating anemia for over 20 years, such agents are often ineffective at treating certain deficiencies in hemoglobin production. Deficiencies in hemoglobin production are often associated with inadequate availability of iron for erythropoiesis, and can result in reduced levels of reticulocyte hemoglobin content (CHr).

Reticulocytes are the youngest erythrocytes released from the bone marrow and circulating in the blood. Within 1-2 days after release from the bone marrow, a circulating reticulocyte matures into a RBC. Due to the rapid turnover of circulating reticulocytes, they represent the current state of RBC production in the body. Reticulocyte hemoglobin content (CHr) provides a convenient measure of effective erythropoiesis and allows a therapy to be adjusted and monitored for achieving maximal treatment efficacy for anemia within a patient. A therapeutic agent that can increase CHr to normal levels and maintain CHr within normal parameters over time, particularly when stimulating hematocrit or hemoglobin increase, would be desirable for effective treatment of anemia.

There remains a need for methods effective at treating deficiencies in hemoglobin production and for increasing reticulocyte hemoglobin content. The present invention meets this need by providing methods and compounds useful for increasing reticulocyte hemoglobin content and treating deficiencies in hemoglobin production.

### SUMMARY OF THE INVENTION

The present invention provides an efficacious therapeutic compound for treating a subject having or at risk for having reduced reticulocyte hemoglobin content. In one embodiment, the invention comprises the compound [(4-hydroxy-1-methyl-7-phenoxy-isoquinoline-3-carbonyl)-amino]-acetic acid for use in methods of increasing or maintaining reticulocyte hemoglobin content (CHr) in a subject in need thereof In a preferred embodiment, the subject is a human subject.

In various embodiments, the invention provides a method for increasing CHr in a subject, the method comprising administering to the subject an effective amount of the compound [(4-hydroxy-1-methyl-7-phenoxy-isoquinoline-3-carbonyl)-amino]-acetic acid. The subject can be a subject having a lower than normal CHr, or any subject in need of increased CHr. In particular embodiments, the subject is a human subject and has a CHr value of less than 28 pg/cell, more particularly less than 26 pg/cell, prior to treatment initiation. In other embodiments, the subject has a CHr between 28 pg/cell and 32 pg/cell, and treatment maintains CHr within the normal range while hemoglobin levels are increased. In various embodiments, the subject's CHr is increased to or maintained within a value of from about 26 pg/cell to about 32.1 pg/cell.

In some embodiments, the compound is administered to the subject two times per week (BIW); in other embodiments, the compound is administered to the subject three times per week (TIW); in other embodiments, the compound is administered to the subject once per week (QW). In various embodiments, compound is administered on alternate days BIW or TIW, or administered QW, at a dose of about 2 milligrams compound per kilogram subject body weight (mg/kg), particularly 1.5 mg/kg, and more particularly 1 mg/kg. In other embodiments, compound is administered on alternate days BIW or TIW, or administered QW, to adult human subjects in an absolute dosage of about 20-400 mg, more particularly 50-100 mg. In preferred embodiments, the compound is administered without administration of supplemental IV iron. In other embodiments, the compound is administered with oral iron supplement.

In various embodiments, the compound is used to treat any subject having a lower than normal CHr or any subject in need of increased CHr, and is particularly for use in subjects having kidney dysfunction. In various embodiments, the subject having a lower than normal CHr or in need of increased CHr has chronic kidney disease, particularly stage 3 or stage 4 chronic kidney disease. In various embodiments, the subject has stage 5 chronic kidney disease or end-stage renal disease. In some embodiments, the subject is not receiving dialysis. In other embodiments, the subject is receiving stable maintenance hemodialysis. In particular embodiments, the subject has been receiving stable maintenance hemodialysis for at least 4 months. In some embodiments, the subject is receiving peritoneal dialysis.

These and other embodiments of the present invention will readily occur to those of skill in the art in light of the disclosure herein, and all such embodiments are specifically contemplated.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including” or “comprising,” or “having,” “containing,” or “involving,” and variations thereof herein, is meant to encompass the items listed thereafter and equivalents thereof as well as additional items.

### BRIEF DESCRIPTION OF THE DRAWINGS

FIG. 1 sets forth data showing methods of the present invention increased reticulocyte hemoglobin content in human subjects with end-stage renal disease.

FIG. 2 sets forth data showing methods of the present invention increased hemoglobin levels in human subject with end-stage renal disease.

FIGS. 3A and 3B sets forth data showing methods of the present invention initially increase and then maintain reticulocyte hemoglobin content in human subjects with chronic kidney disease.

### DESCRIPTION OF THE INVENTION

Before the present compositions and methods are described, it is to be understood that the invention is not limited to the particular methodologies, protocols, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to a “compound that inhibits hypoxia-inducible factor (HIF) prolyl hydroxylase enzyme activity” may be a reference to one or more compounds that inhibits the activity of a hypoxia-inducible factor prolyl hydroxylase enzyme, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

The section headings are used herein for organizational purposes only, and are not to be construed as in any way limiting the subject matter described herein.

### DETAILED DESCRIPTION

The present invention relates generally to methods and medicaments useful for treating a deficiency in hemoglobin production. In particular, methods and compounds for increasing reticulocyte hemoglobin content (CHr) in a subject are provided.

In one embodiment, the invention provides a method for treating a deficiency in hemoglobin production in a subject, the method comprising administering to the subject an effective amount of a compound that inhibits hypoxia-inducible factor (HIF) prolyl hydroxylase enzyme activity, thereby treating the deficiency in hemoglobin production in the subject. In one embodiment, the compound that inhibits HIF prolyl hydroxylase enzyme activity is [(4-hydroxy-1-methyl-7-phenoxy-isoquinoline-3-carbonyl)-amino]-acetic acid. Therefore, methods for treating a deficiency in hemoglobin production in a subject, the methods comprising administering to the subject an effective amount of [(4-hydroxy-1-methyl-7-phenoxy-isoquinoline-3-carbonyl)-amino]-acetic acid, thereby treating the deficiency in hemoglobin production in the subject, are specifically contemplated.

In some embodiments, the deficiency in hemoglobin production is reduced reticulocyte hemoglobin content (i.e., reticulocyte hemoglobin content below normal levels). Reticulocyte hemoglobin content (CHr) is a measure of the amount of hemoglobin in reticulocytes. Reticulocytes are the most immature red blood cells found in circulation in the body and exist in circulation for only a day or two before becoming fully mature red blood cells. Measurement of CHr provides a snapshot of the level of iron directly available for hemoglobin production and is thus an early detector of the body's iron status. In healthy human adults, a normal value for CHr falls within the range of about 24.5-31.8 pg/cell. A CHr value of less than 26 pg/cell may be indicative of iron deficiency, including functional iron deficiency.

The CHr value is thus an index of iron status in an individual and reflects iron status at the level of the erythrocyte. CHr provides an indirect measure of the functional iron available for new red blood cell production over the previous 3-4 days, and is a useful marker of iron deficiency, in particular functional iron deficiency, because it directly reflects iron-deficient erythropoiesis at the erythrocyte level regardless of total body iron stores.

Accordingly, in certain embodiments, the invention provides a method for increasing reticulocyte hemoglobin content (CHr) in a subject, the method comprising administering to the subject an effective amount of a compound that inhibits hypoxia-inducible factor (HIF) prolyl hydroxylase enzyme activity, thereby increasing CHr in the subject. In one embodiment, the compound that inhibits HIF prolyl hydroxylase enzyme activity is [(4-hydroxy-1-methyl-7-phenoxy-isoquinoline-3-carbonyl)-amino]-acetic acid, and methods for increasing CHr in a subject, the method comprising administering to the subject an effective amount of [(4-hydroxy-1-methyl-7-phenoxy-isoquinoline-3-carbonyl)-amino]-acetic acid, thereby increasing CHr in the subject, are thus expressly provided for herein.

In various embodiments of the present invention comprising administering a compound that inhibits HIF prolyl hydroxylase enzyme activity, the administration is oral administration.

It is noted that the methods provided by the present invention are effective at treating a deficiency in hemoglobin production or at increasing CHr in a subject without IV iron supplementation (i.e., without administration of supplemental IV iron to the subject). However, it is contemplated that the present methods can include a combinatorial therapy, i.e., a therapy comprising administering a compound of the invention and further comprising administration of another therapeutic agent, for example, erythropoietin or any other erythropoietic stimulating agent, iron (e.g., oral iron or IV iron), vitamins (e.g., B vitamins), etc.

In a particular embodiment, the present invention provides an efficacious therapeutic compound for treating a subject having reduced reticulocyte hemoglobin content. Specifically, the invention comprises the compound [(4-hydroxy-1-methyl-7-phenoxy-isoquinoline-3-carbonyl)-amino]-acetic acid for use in methods of increasing reticulocyte hemoglobin content (CHr) in a subject. Increases in CHr were observed in human patients treated with compound of the invention, particularly in patients having chronic kidney disease. Data indicate that tested doses between 0.7 and 2.5 milligrams compound per kilogram subject body weight (mg/kg) administered TIW, BIW, or QW were safe, useful, and effective.

Compounds and Medicaments

A preferred compound for use in the present invention is [(4 hydroxy-1-methyl-7-phenoxy-isoquinoline-3-carbonyl)-amino]-acetic acid (Compound A). The compound is one of many compounds generally disclosed in International Publication No. 2004/108681 and U.S. Pat. No. 7,323,475, which provides methods of manufacturing the compounds disclosed therein. However, no particular emphasis was given to this compound in that publication. The present invention demonstrates for the first time that [(4 hydroxy-1-methyl-7-phenoxy-isoquinoline-3-carbonyl)-amino]-acetic acid increases reticulocyte hemoglobin content in human subjects, thereby providing efficacious treatment to patients in need.

The compound may be used in the manufacture of a medicament by means well-known in the art. (See, e.g., Gennaro, ed. (2000) Remington's Pharmaceutical Sciences, supra; and Hardman, Limbird, and Gilman, eds. (2001) The Pharmacological Basis of Therapeutics, supra.) Although the compound may be administered by any suitable route known by those of skill, in preferred embodiments the compound is administered orally.

Pharmaceutical dosage forms of the compound may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of the compound may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the medicament can include one or more physiologically acceptable inactive ingredients that facilitate processing of active molecules into preparations for pharmaceutical use.

For oral administration, the compound can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule. In one embodiment, medicaments include compound in lactose monohydrate and magnesium stearate enclosed in a hypromellose (HPMC) capsule.

The preferred dose for oral administration of compound is between 0.7 and 4.0 milligrams compound per kilogram subject body weight (mg/kg), inclusive. More preferred dose for oral administration is between 1.0 mg/kg and 2.0 mg/kg, or 1.0 mg/kg, 1.5 mg/kg or 2.0 mg/kg.

The compound is particularly efficacious when administered to a subject two times per week (BIW) or three times per week (TIW); in other embodiments, the compound may be administered to the subject once per week (QW). In various embodiments, compound is administered on alternate days BIW or TIW, or administered QW, at a dose of about 2 mg/kg, particularly about 1.5 mg/kg, and more particularly about 1 mg/kg. In other embodiments, compound is administered on alternate days BIW or TIW, or administered QW, to adult human subjects in an absolute dosage of about 20-400 mg, more particularly 50-100 mg. In preferred embodiments, the compound is administered without administration of supplemental IV iron. In some embodiments, the compound is administered with oral iron supplement.

The medicament may, if desired, be presented in a pack or dispenser device containing one or more unit dosage forms containing the compound. Such a pack or device may, for example, comprise metal or plastic foil, such as a blister pack, or glass and rubber stoppers such as in vials. The pack or dispenser device may be accompanied by instructions for administration. Medicaments comprising the compound formulated in a compatible pharmaceutical carrier may also be prepared, placed in an appropriate container, and labeled for treatment of the indicated condition.

Subjects

Subjects suitable for treatment with the methods and compounds of the present invention include any mammalian subjects, such as, but not limited to, human, non-human primate, sheep, horse, cattle, goat, pig, dog, cat, rabbit, guinea pig, hamster, rat, and mouse subjects. In certain embodiments, the subject is a human subject. The subject can be a subject having a deficiency in hemoglobin production, including, for example, a subject having a lower than normal CHr, a subject having anemia, or any subject in need of increased CHr. A subject having anemia will be one having a lower than normal hemoglobin level prior to treatment with the methods and compounds of the present invention. Normal hemoglobin levels for various mammalian species are well known in the art. In particular, for humans, normal hemoglobin levels range from 13 g/dL-18 g/dL for males and 12 g/dL-16 g/dL for females. A human subject having mild to moderate anemia will typically have a hemoglobin level of between 10-12 g/dL, typically between 10-11 g/dL, prior to treatment in the method of the invention. Severely anemic subjects can have hemoglobin levels below 10 g/dL, or below 8 g/dL, or below 6 g/dL.

Anemia can be associated with or result from a number of other conditions or disorders, including but not limited to, chronic kidney disease, end-stage renal disease, cancer, inflammation, infection, and iron deficiency, including absolute iron deficiency and functional iron deficiency, etc., and can afflict subjects undergoing certain treatments, e.g., dialysis, chemotherapy, etc. Accordingly, a suitable subject for the methods of the present invention includes a subject having chronic kidney disease, a subject having end-stage renal disease, a subject having cancer, a subject having iron deficiency, a subject experiencing inflammation or infection, or a subject undergoing a treatment such as dialysis or chemotherapy.

In preferred embodiments, the invention provides a method for increasing reticulocyte hemoglobin content (CHr) in a subject, the method comprising administering to the subject an effective amount of [(4 hydroxy-1-methyl-7-phenoxy-isoquinoline-3-carbonyl)-amino]-acetic acid. The subject can be a subject having a lower than normal CHr, or any subject in need of increased CHr.

Reticulocyte hemoglobin content can be measured by any means known to those of skill in the art. (See, e.g., Mast et al. (2008) Am J Hematol 83:307-310.) Baseline CHr levels may be measured prior to treatment and periodically, e.g., weekly, throughout the treatment period. Normal limits for CHr range from 24.5 pg/cell to 31.8 pg/cell. Values of less than 26 pg/cell are indicative of a patient in need of treatment, particularly for subjects on dialysis. In particular embodiments, the subject is a human subject and has a CHr value of less than 28 pg/cell, particularly less than 26 pg/cell, and more particularly less than 24.5 pg/cell prior to treatment initiation. In various embodiments, the compound [(4 hydroxy-l-methyl-7-phenoxy-isoquinoline-3-carbonyl)-amino]-acetic acid increases to or maintains within normal levels CHr in an anemic patient while hematocrit or hemoglobin is being increased. Thus, in other embodiments, the subject has a CHr between 28 and 32 pg/cell, and treatment maintains CHr within the normal range while hemoglobin levels are increased. In various embodiments, the subject's CHr may be increased to or maintained within a value of from about 26 pg/cell to about 32.1 pg/cell.

The compound is for use in treating any subject having a lower than normal CHr or otherwise in need of increased CHr, and is particularly for use in subjects having kidney dysfunction. In various embodiments, the subject having a lower than normal CHr or in need of increased CHr has chronic kidney disease, particularly stage 3 or stage 4 chronic kidney disease. In various embodiments, the subject has stage 5 chronic kidney disease or end-stage renal disease. In some embodiments, the subject is not receiving dialysis. In other embodiments, the subject is receiving stable maintenance hemodialysis. In particular embodiments, the subject has been receiving stable maintenance hemodialysis for at least 4 months. In some embodiments, the subject is receiving peritoneal dialysis. Dialysis may be carried out in a medical facility or the subject may be receiving home dialysis.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### Increased Reticulocyte Hemoglobin Content (CHr) in Human Subjects with End-Stage Renal Disease

The effect of a compound of the present invention on CHr was determined in human subjects with end-stage renal disease as follows. In the current studies, subjects had been receiving stable maintenance hemodialysis for at least 4 months prior to the initiation of the present study. Additionally, subjects had been maintained on standard of care stable doses of epoetin alfa for the treatment of anemia prior to initiation of the present study.

In order to examine differences in the CHr response in subjects treated with compound of the invention compared to the CHr response in subject treated with epoetin alpha (the current standard of care), all IV iron supplementation was halted two weeks prior to treatment initiation. Administration of oral iron was allowed.

Subjects were administered either compound A (in three treatment groups: 1.0 mg/kg, 1.5 mg/kg, or 2.0 mg/kg) or epoetin alfa thrice-weekly (TIW) for 6 weeks. Baseline CHr levels were determined prior to treatment (mean of two screening period measurements with the Day 1 measurement). CHr levels were measured weekly throughout the six-week study period.

Table 1 below shows the mean baseline CHr and the mean change in CHr after six weeks (43 days) of treatment with three different doses of Compound A (1.0, 1.5, and 2.0 mg/kg TIW) in comparison to TIW treatment with standard of care doses of epoetin alfa. As shown in Table 1, CHr increased following administration of Compound A for 6 weeks. Each dose of Compound A resulted in an increase in CHr. In contrast, CHr decreased in subjects treated with epoetin alfa during the same time period.

TABLE 1

| | | Mean Change |
|---|---|---|
| | | (pg) from |
| | | Baseline CHr at |
| | Mean Baseline | Day 43 (6 weeks) |
| Treatment Group | CHr (pg/cell) | of Treatment |
| Compound A; 1 mg/kg TIW; N = 10 | 31.5 | +0.41 |
| Compound A; 1.5 mg/kg TIW; N = 9 | 31.7 | +0.10 |
| Compound A; 2.0 mg/kg TIW; N = 9 | 32.6 | +0.44 |
| Control: Epoetin alfa; N = 10 | 31.7 | −0.58 |

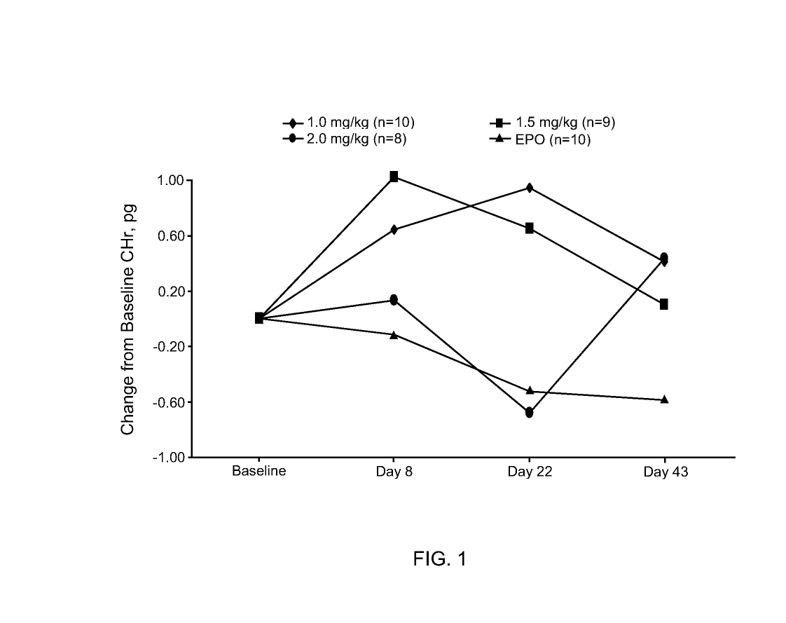

FIG. 1 shows the change (i.e., delta) from baseline of the mean CHr over time during the six week treatment period for each of the three treatment groups treated with Compound A compared to the change from baseline of the mean CHr over the same period in subjects treated with epoetin alfa. As shown in FIG. 1, administration of Compound A at 1.0 mg/kg, 1.5 mg/kg, and 2.0 mg/kg resulted in an increase in mean CHr over time. In contrast, mean CHr decreased over time in subjects administered epoetin alfa.

Additional cohorts dosed with 1.3 mg/kg TIW (N=5) and 1.8 mg/kg TIW (N=5) showed a mean increase in CHr of 0.85 pg/cell and 0.82 pg/cell, respectively, over baseline after 6 weeks of treatment.

These results showed that methods and compounds of the present invention are effective at increasing CHr (to levels above that observed at baseline) in human subjects with end-stage renal disease.

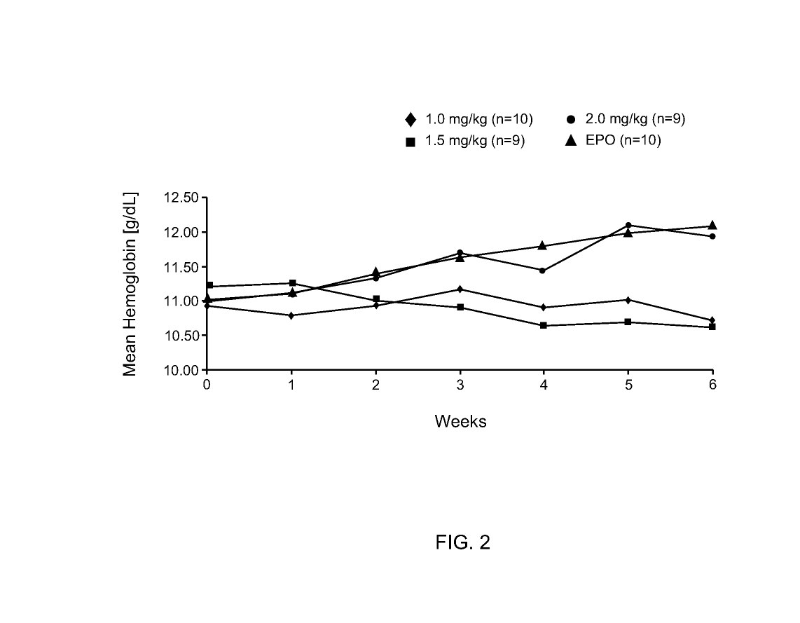

FIG. 2 shows changes in mean hemoglobin levels in the subjects during the six week treatment period. As seen in FIG. 2, administration of Compound A (1.5 mg/kg and 2.0 mg/kg) resulted in a time-dependent increase in mean hemoglobin levels, while administration of epoetin alfa did not result in an increase hemoglobin levels. These results indicated that the present methods and compounds are effective at increasing hemoglobin levels in human subjects with end-stage renal disease, further providing an effective treatment for anemia.

Taken together, these results indicated that administration of Compound A increased CHr in human subjects. As no IV iron was administered to any subjects throughout the six week study period, these results indicated that methods for increasing CHr, as provided by the present invention, are effective at increasing CHr without administration of supplemental IV iron.

### Example 2

### Increased Reticulocyte Hemoglobin Content (CHr) in Human Subjects with Chronic Kidney Disease

The effect of compound of the present invention on CHr was determined in human subjects with stage 3 and stage 4 chronic kidney disease patients (eGFR<60 ml/min) as follows. Subjects with stage 3 or stage 4 chronic kidney disease not receiving dialysis were administered Compound A at either approximately 1.2 mg/kg twice weekly or 70 mg fixed dose thrice weekly for a period of 17 weeks. Dose adjustments were permitted from week 5 onward to correct and maintain subjects to a target hemoglobin range; however, doses were not allowed to exceed 2.5 mg/kg per dose. CHr values were measured at week 9 and week 17.

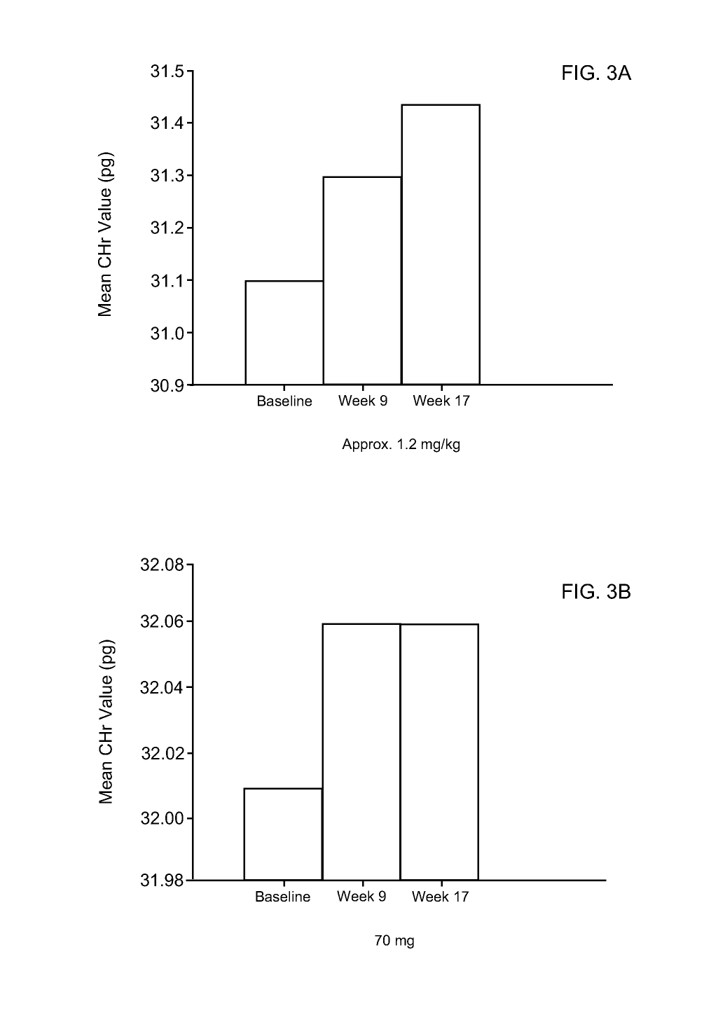

As shown in FIG. 3, mean CHr increased over time in subjects administered Compound A at a dose of either approximately 1.2 mg/kg BIW (FIG. 3A) or 70 mg TIW (FIG. 3B). FIG. 3B also shows that fixed dosing demonstrated maintenance of mean CHr from 9 to 17 weeks. These results indicated that methods and compounds of the present invention are effective at increasing CHr and maintaining CHr within normal ranges during correction of hemoglobin levels in human subjects with stage 3 or stage 4 chronic kidney disease.

1. A method of increasing reticulocyte hemoglobin content (CHr) in a subject, the method comprising administering the compound [(4-hydroxy-1-methyl-7-phenoxy-isoquinoline-3-carbonyl)-amino]-acetic acid to the subject, thereby increasing CHr.

2. The method of claim 1, wherein the subject has a lower than normal CHr.

3. The method of claim 1, wherein the subject is in need of increased CHr.

4. The method of claim 1, wherein the subject is a human subject.

5. The method of claim 4, wherein the subject has a CHr value of less than 28 pg/cell.

6. The method of claim 1, wherein the method does not include administration of supplemental IV iron to the subject.

7. The method of claim 1, wherein the subject has chronic kidney disease.

8. The method of claim 7, wherein the subject has stage 3 or stage 4 chronic kidney disease.

9. The method of claim 1, wherein the subject has end-stage renal disease.

10. The method of claim 9, wherein the subject is receivinge stable maintenance hemodialysis.
